(19) United States
(12) Patent Application Publication
(10) Pub. No.: US 2020/0258625 A1
(43) Pub. Date: Aug. 13, 2020

(54) SERVER-NEUTRAL NETWORK ARCHITECTURE

(71) Applicant: Darroch Medical Solutions, Inc., San Diego, CA (US)

(72) Inventors: Colby Bishop, San Diego, CA (US); Anthony Shao, Hillsborough, CA (US); Jacques Yeager, Riverside, CA (US); Subramanian Shastri, Carlsbad, CA (US)

(21) Appl. No.: 16/784,727

(22) Filed: Feb. 7, 2020

Related U.S. Application Data
(60) Provisional application No. 62/804,374, filed on Feb. 12, 2019.

(51) Int. Cl. G16H 40/67; G16H 10/60

(52) U.S. Cl. CPC G16H 40/67; G16H 10/60

(57) ABSTRACT

Computer-implemented server-neutral communications architecture and method of analyzing, authenticating and transmitting medical data independent of a server are provided. Exemplary server-neutral communications architecture includes a communication device including a housing and a transceiver circuit, a processor, and a battery disposed within the housing, one or more medical instruments, and a data analytics system. The medical instruments collect data from a medical patient and transmit the data relating to the medical patient directly to the communication device. The data analytics system determines which data come from a particular medical instrument. Advantageously, the medical instruments communicate directly with the communication device independent of transmitting the data through a server. The communication device communicates information about a patient's medical condition directly to a mobile device or a workstation. Disclosed systems and methods may develop a patient proxy based upon the medical data and communicate the patient proxy directly to a mobile device or a workstation in real time

### CROSS-REFERENCE TO RELATED APPLICATION

This application claims priority to and benefit of U.S. Patent Application No. 62/804,374, filed Feb. 12, 2019, which is hereby incorporated by reference in its entirety.

### FIELD OF THE DISCLOSURE

The present disclosure relates to server-neutral network architecture that provides medical practitioners with bedside treatment and medical device data, patient progression, and patient-specific analytics information in real time.

### BACKGROUND

In current hospital information technology (IT) systems, there are two methods by which medical instrument data is sent to Electronic Medical Record (EMR) systems. The first involves manual entry by nurses using workstations at a nurses' station. However, this method is extremely tedious, prone to errors, and does little to improve nurse-patient interactions.

The second method is through the use of vendor-specific servers, which communicate with all instruments supported by the vendor. Servers have been in place as a means of aggregating large amounts of data for any purpose; however, due to the nature of the medical field and the sheer amount of data, much of the information gathered is of little use. Also, in the current hospital architecture, there is no “gateway” to monitor and filter what information is pushed to the EMR, and because of this there have been few efforts to coordinate the information.

Data is automatically transmitted from instruments or medical devices to the servers using the hospital network. Unfortunately, virtually all of this data stays in the server or is discarded until an instrument specific alarm or a patient specific event occurs; an example of this could be if a patient's blood pressure suddenly drops below predetermined levels or if a patient stops receiving an infusion.

There are several problems with this method of data communication. First and foremost, vendor specific servers are costly to implement due to a requirement that multiple vendor specific servers will need to be implemented and supported if there are multiple medical devices from separate vendors. A typical hospital may consist of 250 rooms or more, with each room housing several medical instruments, each attempting to connect to its company-specific server. The resulting cost of establishing and maintaining a communication network could be as high as hundreds of thousands of dollars. Further, even with the investment, most of the patient data is inaccessible to medical practitioners' unless they are at designated workstations.

A second problem with the server architecture is that there are significant latencies in the transmission of data from instruments to medical practitioners. Most instruments send information to their respective servers in batches, often a few times a day. Information that is stored in these servers is also only transferred to the EMR at designated times during a day as well, usually once a day at night. Hospitals could increase the frequency in which medical data is sent from server to EMR, but with so many company-specific servers in place, each with their own information protocols, it becomes nearly impossible for a cost-effective network to accommodate. This means that EMRs may take several hours to update information at a nurse's station, which in turn would render patient data too outdated to be of use in treatment. Even worse, many medical practitioners have claimed that they elect to manually chart data due to the frustration of trying to learn how to properly use EMRs.

A third problem is that patient data from medical instruments is available mostly only at a nurse's station. Unfortunately, nurses are rarely at their station during shifts and are instead focused on providing care to their patients. This results in a mismatch of information between practitioners who are making rounds and those who are monitoring the EMRs.

Thus, it becomes readily apparent that there exists a great need for information to be presented to medical practitioners in a timely manner and at their location, not just at workstations. There is also a need for systems and methods of delivering medical data without a server-based network architecture. Medical data is extremely important in showing the progression of a patient's health and can even be used to predict adverse events as well as a patient's potential recovery. But today's information architecture does not provide the right infrastructure for this information to be properly delivered to a nurse practitioner or doctor without introducing significant additional computer hardware, network bandwidth and data latency.

### SUMMARY

The present disclosure, in its many embodiments, alleviates to a great extent the disadvantages of server-based architecture for transmitting medical data. Disclosed embodiments provide a way to deliver medical instrument data to medical practitioners reliably and in a timely manner without the need for a server-based architecture. Using any form of device-to-device communications (e.g. Wi-Fi, Bluetooth, ZigBee), the Care Life Unit (CLU) consists of a means of acquiring data directly from medical instruments by a patient bedside, aggregating the data thus acquired to develop a short-term profile of a patient's medical condition, and delivering both the data and the profile to a medical practitioner in a manner that is easily used and manipulated.

We refer to the information and analyses presented to the medical practitioner as a patient proxy, implying that the CLU presents the practitioner a medical proxy representation of the patient. With the patient proxy, the practitioner would see all the patient information in one centralized location in real time. In exemplary embodiments, each bed will have one CLU, tagged to a specific patient and corresponding with the patient's medical practitioner.

Instead of medical devices connecting only to their vendor-specific servers, devices will instead also connect to the CLU. The CLU, acting as an information intermediary, will be able to organize the raw data that comes in from each of the medical instruments. It will then directly communicate the patient profile to a mobile device that can be carried by a medical practitioner.

By not relying on servers, information can be pushed from device to practitioner in real time. Further, it moves the hospital architecture away from one that centralizes the collection of data from all instruments supported by a vendor over the hospital network, to one that collects data from multiple instruments by the bedside and aggregates data into a single patient profile and communicates patient profile over the hospital network. We call an architecture that supports real time device-to-device communication without the use of servers of patient profile as a “server-neutral” architecture, and state this as a primary invention claimed in this document.

In addition to mobile devices, the system will have the ability to push patient profiles to a nurse's station. The user interface available in the mobile device and the workstation will have an EMR update function. All this will facilitate redundancy in the availability of information and provide a failsafe mechanism in the mitigation of adverse events during hospitalization. We call the architecture that combines device-to-device communication with traditional server-based communication as Server Neutral Architecture.

Exemplary embodiments of a computer-implemented server-neutral communications architecture comprise a communication device including a housing and a transceiver circuit, a processor, and a battery disposed within the housing, one or more medical instruments, and a data analytics system. The medical instruments collect data from a medical patient and transmit the data relating to the medical patient directly to the communication device. The data analytics system determines which data come from a particular medical instrument. The one or more medical instruments may include infusion pumps, vital signs monitors, and/or bed scales. The medical instruments communicate directly with the communication device independent of transmitting the data through a server. The communication device communicates information about a patient's medical condition directly to a mobile device or a workstation. The communications architecture may include a user interface viewable on a mobile device.

In exemplary embodiments, the information about a patient's medical condition is communicated to a mobile device or a workstation in real time. The information about a patient's medical condition may be based on determinations of the data analytics system. In exemplary embodiments, the data analytics system determines whether a patient may experience an adverse event through comparisons between the patient's progression and trends and an adverse event repository. The communications architecture may cause an alarm to sound when the data relating to the medical patient is outside pre-determined boundary parameters or a medical instrument has stopped functioning or changed its rate, or a patient may experience an adverse event.

In exemplary embodiments, there are a plurality of communication devices and each of the plurality of communication devices collects data from a different medical patient. In exemplary embodiments, each of the plurality of communication devices is in communication with the same mobile device or workstation. The medical instruments may communicate directly with the communication device without transmitting the data through a server.

Exemplary computer-implemented methods of analyzing, authenticating and transmitting medical data independent of a server comprise transmitting medical data from one or more medical instruments directly to a communication device independent of transmitting the data through a server, developing a patient proxy based upon the medical data, and communicating the patient proxy directly to a mobile device or a workstation in real time. Developing a patient proxy may comprise creating one or more boundary parameters for the data and determining whether any data is outside the boundary parameters. In exemplary embodiments, developing a patient proxy further comprises creating trends based on the aggregated data and comparing trends to existing adverse event symptoms. Exemplary methods may further comprise sounding an alarm when selected data is outside pre-determined boundary parameters or a particular medical instrument has stopped functioning, changed its rate, or if a patient may experience an adverse event. In exemplary embodiments, a plurality of prioritizable alarms are provided.

Exemplary methods further comprise displaying room numbers or bed numbers corresponding to patients. Disclosed methods may further comprise displaying patient vital signs data, other measured metrics, or a patient's progression relating to a vital sign. Exemplary methods comprise presenting a color scheme wherein different colors represent how a patient is progressing. In exemplary embodiments, the communication device comprises a plurality of communication devices, each of the plurality of communication devices collects data from a different medical patient, and each of the plurality of communication devices is in communication with the same mobile device or workstation.

It is an object of disclosed embodiments to wirelessly acquire and analyze data from at least three treatment and monitoring devices (i.e. Infusion Pumps, Vital Signs Monitors, and Bed Scales) and communicate a cohesive patient profile with a handheld device through the mobile application independent of a vendor-specific server. It is an object of disclosed embodiments to wirelessly acquire and analyze data from treatment and monitoring devices attached to a hospitalized patient and communicate changes in the patient's condition to a nurse's handheld device in a live hospital environment. It is an object of disclosed embodiments to wirelessly connect to the three bedside instruments, analyze incoming data, and push all relevant patient data and potential onset adverse event information to the healthcare practitioner in both simulated and live environments.

Accordingly, it is seen that systems and methods of analyzing, authenticating and transmitting medical data independent of a server are provided. These and other features and advantages will be appreciated from review of the following detailed description, along with the accompanying figures in which like reference numbers refer to like parts throughout.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a schematic of an exemplary embodiment of a server-neutral communication architecture in accordance with the present disclosure; and

FIG. 2 is a process flow diagram of an exemplary embodiment of a method of analyzing, authenticating and transmitting medical data independent of a server in accordance with the present disclosure.

### DETAILED DESCRIPTION

In the following detailed description of exemplary embodiments of the disclosure, reference is made to the accompanying drawings in which like references indicate similar elements, and in which is shown by way of illustration specific embodiments in which disclosed systems and devices may be practiced. These embodiments are described in sufficient detail to enable those skilled in the art to practice the embodiments, and it is to be understood that other embodiments may be utilized and that logical, mechanical, functional, and other changes may be made without departing from the scope of the present disclosure. The following detailed description is, therefore, not to be taken in a limiting sense, and the scope of the present disclosure is defined by the appended claims. As used in the present disclosure, the term “or” shall be understood to be defined as a logical disjunction and shall not indicate an exclusive disjunction.

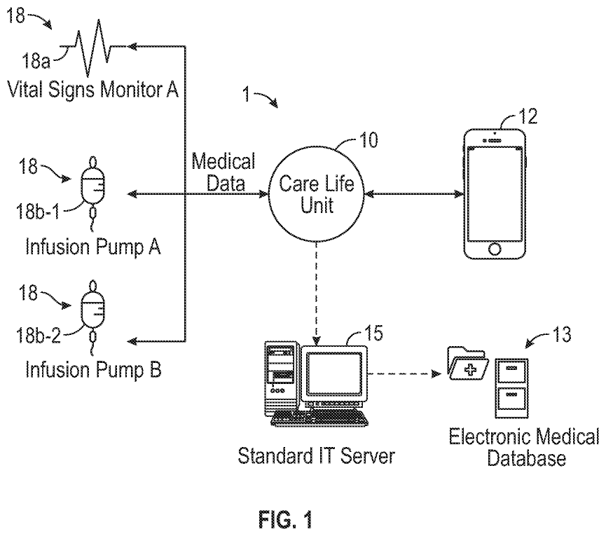

FIG. 1 is a high-level schematic of exemplary server-neutral communications architecture 1 that collects, aggregates, analyzes, and transmits medical information. Disclosed embodiments comprise hardware, software and firmware. More particularly, a communication device 10 (the CLU mentioned above), as described in detail in co-pending U.S. patent application Ser. No. 15/962,290, filed Apr. 25, 2018, and hereby incorporated by reference in its entirety, has a transceiver (not shown) and circuit (not shown) having a communication channel. The device 10 has a processor (not shown) and memory (not shown) in communication with the transceiver circuit. Typically, embodiments will have one transceiver circuit including one transceiver, but some embodiments may have a second transceiver circuit.

Communication device 10 receives medical data from at least one medical instrument 18, which may be equipped with a transceiver or dongle. The medical instrument could be a vital-signs monitor 18a, an infusion pump 18b-1, 18b-2, or any other medical instrument that monitors or provides medication or treatment to patients. In the current hospital environment, medical devices (such as infusion pumps) take pertinent patient readings and measurements. In exemplary embodiments, there are three medical instruments or types of instruments 18 (Infusion Pumps, Vital Signs Monitors, and Bed Scales). Of course, there are other bedside treatment and medical devices that can be integrated in the future.

The CLU or communication device 10 acts as the gateway. Because the purpose of the CLU is to present practitioners with relevant data quickly, vendor-specific servers do not need to be used. Instead, because the CLU pushes short-term patient profiles to practitioners, opting for serverless communications would be one of the most viable methods in pushing data reliably and in a timely manner. A standard IT server may be implemented as a second failsafe to ensure patient proxy updates are not lost if an outage occurs; however, it is not essential to the server-neutral architecture.

The primary function of communication device 10 is to act as an information intermediary or gateway in which filtered data from medical instruments 18 can be passed directly from the medical instruments to the communication device 10 and from the communication device to a mobile interface. The communication device 10 itself is a microcomputer that has the ability to receive and transmit data, process and manipulate information, and store it for future use. One communication device can connect to up to eight medical instruments, and in exemplary embodiments there will be one communication device per hospital bed. Since practitioners in non-ICU wards tend to have up to four or five patients, several communication devices can connect to one mobile interface; in this manner, medical practitioners can remotely monitor several of their patients at once.

To ensure that the information is passed correctly from medical devices 18a, 18b-1, 18b-2 to the communication device 10, medical devices will first securely establish communication connections with the communication device. Communication connections can employ a variety of methods to connect to the communication device 10, methods that include but are not limited to Bluetooth, Wi-Fi, Cellular, or ZigBee. Once the connection is made, the medical device can begin to stream data. The information that is passed through any of the aforementioned communication protocols will correspond to the appropriate transceiver on the communication device's main circuit board (Bluetooth to Bluetooth, Wi-Fi to Wi-Fi). Communication device 10 will then sort the information based on the medical instrument provided and develop a short-term profile of a patient's medical condition. Finally, the communication device 10 will send the data to a practitioner via a mobile interface (e.g. smartphone, tablet, etc.) to which the he/she can use and manipulate.

Though FIG. 1 only shows one communication device 10 connected, several communication devices can connect to a single practitioner. In this manner, a nurse or doctor can remotely monitor his or her patients from one mobile screen and in real time. This is one of the key advantages to a server neutral architecture: with servers in place, practitioners rely primarily on data that comes from EMRs and medical devices. With the disclosed server neutral architecture and streamlined data protocols in place, a medical practitioner can keep tabs on multiple patients without having to be in the room.

A particularly advantageous feature of disclosed server-neutral architecture 1 and communication device 10 is the ability to transmit medical data to a medical practitioner without the use of company-specific servers. An example of the methodology would be as follows: (1) A medical instrument 18 sends information over Bluetooth to the communication device 10; (2) communication device 10 organizes information based off practitioner input; communication device 10 then sends information over Wi-Fi to a the screen on practitioner's mobile device 12. Information passed from medical instrument 18 to communication device 10 and from communication device to mobile device 12 interface can employ a variety of different communication protocols. Bluetooth and Wi-Fi are some of the more common examples today, but virtually any method of communication is viable so long as the information is provided in a timely manner.

Another feature of exemplary server neutral architecture 1 is that multiple communication devices can also connect to one mobile interface. In this way, one medical practitioner can remotely monitor multiple patients at once. In the current EMR-based monitoring system, practitioners must sit at a nurse's station in order to view any information that is inputted into the system by other practitioners. However, this usually does not include medical device data. In addition, many practitioners have noted how laborious and slow many of these EMR systems have become when trying to view how multiple patients are faring.

One of the more prominent features of disclosed server-neutral architecture 1 and communication device 10 is the ability to transmit data in real-time. Most servers and EMRs only measure data when a sudden artifact in data arises (e.g. blood pressure rises, temperature drops, heart rate increases), but retroactively assessing an artifact in data is useless if the patient's health fails. Having medical device data in real time can be extremely useful in Early Warning Systems. For example, patient falls are some of the most common injuries in hospitals. In 2013, the Agency for Healthcare Research and Quality estimated that 700,000 to 1 million hospitalized patients fall every year. This results in further health care costs that could have been avoided had a practitioner known that a patient has risen from his or her bed. In addition, real time data would be incredibly difficult to implement in server or EMR based architectures because the servers cannot poll medical device data at a fast-enough rate to accommodate all the medical instruments that are being connected. Instead of using a conglomerate server to measure all of the data, the CLU can streamline the information being pushed from medical device to mobile interface, resulting in more effective patient monitoring.

Exemplary embodiments may include an additional backup server 15 that holds information. The communication device 10 can function without the backup server 15 with the ability to push information to medical practitioners, but the server's implementation serves as a means to continue holding data if a mobile device cannot be reached. This can also help with packet loss if the connection between medical instruments or mobile phones is constantly being disrupted. It will be up to the hospital's discretion as to whether they would like to implement the additional server 15.

Other variations to the server neutral architecture are mainly in the form of the medical device 18 to communication device 10 and communication device to mobile interface communications. As discussed above, the main forms of communication will be Bluetooth, Wi-Fi, ZigBee, and hard wiring. However, there can be other forms of communication so long as the information is provided in a timely manner.

The hardware used on the communication device 10 will be enough to handle data input and output, forms of analytics, and data storage; an example of the hardware would be a microprocessor, RAM, batteries, and a microSD card. These are some of the examples that are used in creating the server bypass structure with the communication device 10. The communication device 10 need not have immense computing power but should be enough to handle the aforementioned tasks with little latency.

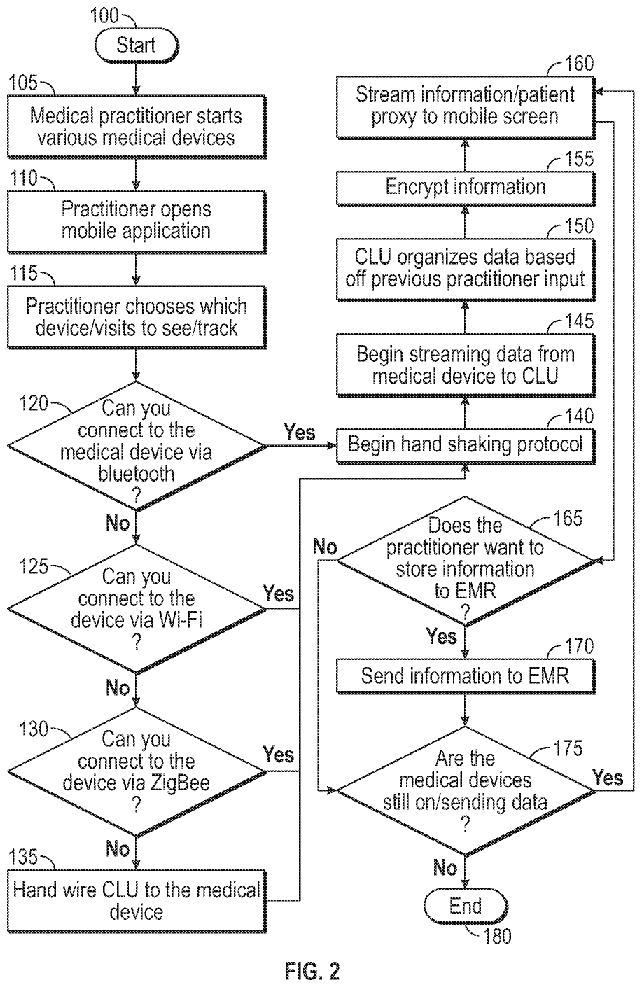

Exemplary server-neutral architecture flow, shown in FIG. 2, illustrates operation. There are various communication protocol attempts via Wi-Fi, Bluetooth, and Zigbee instead of company-specific servers. In addition, the flowchart also shows how the data flows from medical instruments 18 to the communication device 10 and various examples in which practitioners can influence how the data flows. At the start 100, the medical practitioner may start 105 various medical instruments, then open 110 his or her mobile application. The practitioner would choose which device(s)/vitals he or she wants to track 115 and then the communications architecture would query 120 whether the practitioner can connect to the desired medical device(s) via Bluetooth.

If the practitioner cannot connect to the desired medical device(s) via Bluetooth, the communications architecture queries 125 whether connection can be made via Wi-Fi. If a Wi-Fi connection is unavailable, the communications architecture queries 130 whether connection can be made via Zigbee. Finally, if all of those are unsuccessful, the medical practitioner would hard wire 135 the communication device to the desired medical instrument(s). If Bluetooth connection is available (or when the hard wiring is done), the communications architecture begins the hand-shaking protocol 140 and then begins streaming 145 data from the medical device(s) to the communication device.

The communication device organizes 150 the data based upon previous practitioner input and encrypts 155 the data. The communication device will begin to stream 160 medical data and/or a patient proxy to the mobile screen of the medical practitioner. If the practitioner wants to store 165 the information in the EMR database 13, the communications architecture sends 170 the medical data and/or patient proxy to the EMR. If the medical data is not to be stored in the EMR, the communications architecture queries 175 whether the medical instruments are still operating and sending medical data. If so, the medical data and/or patient proxy continues to be sent 160 to the mobile screen of the medical practitioner. If not, the communications operation ends 180.

The concept of a patient proxy, including method of building and presentation, is described in detail in co-pending U.S. patent application Ser. No. 16/594,111, filed Oct. 7, 2019, which is hereby incorporated by reference in its entirety, and will be briefly described here. There are three components or steps to building the patient proxy: data aggregation, data coordination and analysis, and data presentation. As discussed above, server-neutral architecture 1 is the system that enables all the steps including transmitting the patient proxy to medical practitioners. Data aggregation is the first step in creating the patient proxy. It consists of capturing data outputted by any number of medical instruments in real time via any form of device communications (e.g. Wi-Fi, Bluetooth, ZigBee, etc.). Medical instruments are typically designed to output information to the EMRs and need to be redirected towards the communication device 10. Redirection can be done via hardware or software.

The second stage in creating the patient proxy is the data coordination and analysis stage, performed by a data analytics system. The second stage includes parsing through all the information that comes into the communication device 10 and determining which information belongs to which medical instrument 18. Practitioners will also be able to input boundary parameters for any of the statistics that are measured; when any of the measured data do not fit inside the boundary conditions, the nurse will be alerted based on what the software has found. Exemplary embodiments of the software will be able to cross reference medical device data and alarms with vital signs monitor information to give practitioners a better understanding of how their medical devices are affecting the patient in real time.

There are (at least) two pieces that go into the data analytics facet of the patient proxy: the repository of adverse event (AE) symptoms and the algorithms that both disseminate trends (i.e. physiological symptoms) from the measured bedside data and compare said symptoms to the repository. The AE repository serves as the “master-guide” for the different AE that a patient may exhibit; events such as sepsis, infection, and cardiac arrest may be predicted by variations in independent vitals and through the cross examination of multiple vitals together. The repository can be comprised of existing literature (e.g. case studies) and examined data retroactively gathered from AE that occur in the hospital.

The microanalysis algorithms essentially function as a means to which raw patient data, measured from the various bedside medical instruments, can be converted into trends that are compared to the AE repository; an example of this is the transformation of three hours of measured pulse rate data to a potential directional trend and/or comparison to the transformation of another measured metric. The microanalysis algorithms will also then be able to compare the deduced trends to the AE repository, ascertain the potential of certain AEs, and notify the responsible practitioners.

It should be noted that one important difference between the patient proxy and individual device analysis is that non-vital signs monitoring machines will be cross analyzed with vital signs information. In this way, a practitioner now understands how each medical device impacts the overall health and status of the patient through vital signs information in real time.

The third stage in generating a patient proxy is how the information is presented on a mobile device 12. In exemplary embodiments, there are several screens detailing the patient proxy. A patient proxy 1 is a cohesive image of a patient's medical information and progression, which is pushed to a mobile device 12 in real-time. Exemplary embodiments do that through a software application on mobile devices, which, in exemplary embodiments has at least three layers to it: Home Screens, In-Depth Screens, and Graphing Screens. These three screens together are intended to provide the healthcare practitioner a way to understand how his/her patients are recovering without the need to manually inspect the instruments, which if a practitioner has multiple patients, can be incredibly difficult to do. Although a patient proxy is a virtual construct, for purposes of illustration, it can be thought of as the information presented in the three screens.

An exemplary graphical or user interface for the patient proxy displays the history of the patient's progression based on a single metric, which could be the pulse. Exemplary embodiments of a home screen for the mobile application give the healthcare practitioner a quick method to ascertain the status of his or her patients; the first “home” page of a patient proxy is a way for practitioners to quickly see how their patient is doing and shows patients by room or bed number. An exemplary in-depth screen or page might appear when a medical practitioner selects any of the rooms or beds displayed on a home screen. Here, a practitioner would be able to see how all the medical instruments are operating, enter various boundary conditions, and ensure that the patient is within those conditions. The in-depth page also gives a tremendous amount of assistance relating to the onset of potential adverse events.

In addition to presenting information from various medical devices as described above, exemplary patient proxies, systems and methods also feature the ability to input upper and lower boundaries for any of the medical device parameters that are being collected or displayed. This serves as the practitioner's ability to prioritize alarms and reduce alarm fatigue.

While the disclosed systems and devices have been described in terms of what are presently considered to be the most practical exemplary embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

Thus, it is seen that improved server-neutral architecture and associated communication devices, systems, and methods are provided. It should be understood that any of the foregoing configurations and specialized components may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

1. A computer-implemented server-neutral communications architecture comprising:
a communication device including a housing and a transceiver circuit, a processor, and a battery disposed within the housing;
one or more medical instruments collecting data from a medical patient and transmitting the data relating to the medical patient directly to the communication device;
a data analytics system determining which data come from a particular medical instrument;
wherein the medical instruments communicate directly with the communication device independent of transmitting the data through a server; and
wherein the communication device communicates information about a patient's medical condition directly to a mobile device or a workstation.

2. The server-neutral communications architecture of claim 1 wherein the information about a patient's medical condition is communicated to a mobile device or a workstation in real time.

3. The server-neutral communications architecture of claim 1 wherein the information about a patient's medical condition is based on determinations of the data analytics system.

4. The server-neutral communications architecture of claim 1 wherein the data analytics system determines whether a patient may experience an adverse event through comparisons between the patient's progression and trends and an adverse event repository.

5. The server-neutral communications architecture of claim 1 wherein an alarm sounds when the data relating to the medical patient is outside pre-determined boundary parameters or a medical instrument has stopped functioning or changed its rate, or a patient may experience an adverse event.

6. The server-neutral communications architecture of claim 1 wherein the one or more medical instruments include one or more of: infusion pumps, vital signs monitors, and bed scales.

7. The server-neutral communications architecture of claim 1 further comprising a user interface viewable on a mobile device.

8. The server-neutral communications architecture of claim 1 wherein the communication device comprises a plurality of communication devices.

9. The server-neutral communications architecture of claim 8 wherein each of the plurality of communication devices collects data from a different medical patient.

10. The server-neutral communications architecture of claim 9 wherein each of the plurality of communication devices is in communication with the same mobile device or workstation.

11. The server-neutral communications architecture of claim 1 wherein the medical instruments communicate directly with the communication device without transmitting the data through a server.

12. A computer-implemented method of analyzing, authenticating and transmitting medical data independent of a server, comprising:
transmitting medical data from one or more medical instruments directly to a communication device independent of transmitting the data through a server;
developing a patient proxy based upon the medical data; and
communicating the patient proxy directly to a mobile device or a workstation in real time.

13. The computer-implemented method of claim 12 wherein developing a patient proxy comprises creating one or more boundary parameters for the data and determining whether any data is outside the boundary parameters.

14. The computer-implemented method of claim 13 wherein developing a patient proxy further comprises creating trends based on the aggregated data and comparing trends to existing adverse event symptoms.

15. The computer-implemented method of claim 13 further comprising sounding an alarm when selected data is outside pre-determined boundary parameters or a particular medical instrument has stopped functioning, changed its rate, or if a patient may experience an adverse event.

16. The computer-implemented method of claim 12 further comprising displaying room numbers or bed numbers corresponding to patients.

17. The computer-implemented method of claim 14 further comprising displaying patient vital signs data, other measured metrics, or a patient's progression relating to a vital sign.

18. The computer-implemented method of claim 15 further comprising providing a plurality of prioritizable alarms.

19. The computer-implemented method of claim 12 further comprising presenting a color scheme wherein different colors represent how a patient is progressing.

20. The computer-implemented method of claim 12 wherein the communication device comprises a plurality of communication devices, each of the plurality of communication devices collects data from a different medical patient, and each of the plurality of communication devices is in communication with the same mobile device or workstation.
